(19) United States
(12) Patent Application Publication
(10) Pub. No.: US 2004/0110208 A1
(43) Pub. Date: Jun. 10, 2004

(54) Methods and device for DNA sequencing using surface enhanced Raman scattering (SERS)

(76) Inventors: Selena Chan, San Jose, CA (US); Xing Su, Cupertino, CA (US)

Correspondence Address: LISA A. HAILE, Ph.D., ATTORNEY FOR INTEL CORPORATION, GRAY CARY WARE & FREIDENRICH LLP, 4365 Executive Drive, Suite 1100, San Diego, CA, 92121-2133, US

(21) Appl. No.: 10/672,149

(22) Filed: Sep. 26, 2003

Related U.S. Application Data
(63) Continuation-in-part of application No. 10/108,128, filed on Mar. 26, 2002.

(51) Int. Cl. C12Q 1/68

(52) U.S. Cl. 435/6

(57) ABSTRACT

The methods and apparatus disclosed herein concern nucleic acid characterization by enhanced Raman spectroscopy. In certain embodiments of the invention, exonuclease treatment of the nucleic acids results in the release of nucleotides. The nucleotides may pass from a reaction chamber through a microfluidic channel and enter a nanochannel or microchannel. The nanochannel or microchannel may be packed with nanoparticle aggregates containing hot spots for Raman detection. As the nucleotides pass through the nanoparticle hot spots, they may be detected by Raman spectroscopy. Identification of the sequence of nucleotides released from the nucleic acid is used to characterize the nucleic acid, for example by sequencing or identifying the nucleic acid. Other embodiments of the invention concern apparatus for nucleic acid sequencing.

### RELATED APPLICATIONS

The present application is a continuation-in-part of U.S. patent application Ser. No. 10/108,128, filed May 26, 2002.

### FIELD

The present methods, compositions and apparatus relate to the fields of molecular biology and genomics. More particularly, the methods, compositions and apparatus concern nucleic acid characterization by Raman spectroscopy. Characterization may involve identifying or sequencing the nucleic acid.

### BACKGROUND

Genetic information is stored in the form of very long molecules of deoxyribonucleic acid (DNA), organized into chromosomes. The human genome contains approximately three billion bases of DNA sequence. This DNA sequence information determines multiple characteristics of each individual. Many common diseases are based at least in part on variations in DNA sequence.

Determination of the entire sequence of the human genome has provided a foundation for identifying the genetic basis of such diseases. However, a great deal of work remains to be done to identify the genetic variations associated with each disease. That would require DNA sequencing of portions of chromosomes in individuals or families exhibiting each such disease, in order to identify specific changes in DNA sequence that promote the disease. Ribonucleic acid (RNA), an intermediary molecule in processing genetic information, may also be sequenced to identify the genetic bases of various diseases.

Existing methods for nucleic acid sequencing, based on detection of fluorescently labeled nucleic acids that have been separated by size, are limited by the length of the nucleic acid that can be sequenced. Typically, only 500 to 1,000 bases of nucleic acid sequence can be determined at one time. This is much shorter than the length of the functional unit of DNA, referred to as a gene, which can be tens or even hundreds of thousands of bases in length. Using current methods, determination of a complete gene sequence requires that many copies of the gene be produced, cut into overlapping fragments and sequenced, after which the overlapping DNA sequences may be assembled into the complete gene. This process is laborious, expensive, inefficient and time-consuming. It also typically requires the use of fluorescent or radioactive labels, which can potentially pose safety and waste disposal problems.

More recently, methods for nucleic acid sequencing have been developed involving hybridization to short oligonucleotides of defined sequenced, attached to specific locations on DNA chips. Such methods may be used to infer short nucleic acid sequences or to detect the presence of a specific nucleic acid in a sample, but are not suited for identifying long nucleic acid sequences.

### BRIEF DESCRIPTION OF THE DRAWINGS

The following drawings form part of the present specification and are included to further demonstrate certain aspects of the disclosed methods and apparatus. The methods and apparatus may be better understood by reference to one or more of these drawings in combination with the detailed description of specific embodiments presented herein.

FIG. 1 illustrates an exemplary apparatus 100 (not to scale) and method for nucleic acid 109 sequencing by surface enhanced Raman spectroscopy (SERS), surface enhanced resonance Raman spectroscopy (SERRS) and/or coherent anti-Stokes Raman spectroscopy (CARS) detection.

FIG. 2 shows the Raman spectra of all four deoxynucleoside monophosphates (dNTPs) at 100 mM concentration, using a 100 millisecond data collection time. Characteristic Raman emission peaks for as shown for each different type of nucleotide. The data were collected without surface-enhancement or labeling of the nucleotides.

FIG. 3 shows SERS detection of 1 nM guanine, obtained from dGMP by acid treatment according to Nucleic Acid Chemistry, Part 1, L. B. Townsend and R. S. Tipson (Eds.), Wiley-Interscience, New York, 1978.

FIG. 4 shows SERS detection of 100 nM cytosine.

FIG. 5 shows SERS detection of 100 nM thymine.

FIG. 6 shows SERS detection of 100 pM adenine, obtained from DAMP by acid treatment.

FIG. 7 shows a comparative SERS spectrum of a 500 nM solution of deoxyadenosine triphosphate covalently labeled with fluorescein (upper trace) and unlabeled dATP (lower trace). The dATP-fluorescein was obtained from Roche Applied Science (Indianapolis, Ind.). A strong increase in the SERS signal was detected in the fluorescein labeled DATP.

FIG. 8 shows the SERS detection of a 0.9 nM (nanomolar) solution of adenine. The detection volume was 100 to 150 femtoliters, containing an estimated 60 molecules of adenine.

FIG. 9 shows the SERS detection of a rolling circle amplification product, using a single-stranded, circular M13 DNA template.

### DESCRIPTION OF ILLUSTRATIVE EMBODIMENTS

The disclosed methods, compositions and apparatus are of use for the rapid, automated sequencing of nucleic acids. The methods and apparatus may be suitable for obtaining the sequences of very long nucleic acid molecules of greater than 1,000, greater than 2,000, greater than 5,000, greater than 10,000 greater than 20,000, greater than 50,000, greater than 100,000 or even more bases in length. Advantages over prior art methods include the ability to read long nucleic acid sequences in a single sequencing run, greater speed of obtaining sequence data, decreased cost of sequencing and greater efficiency in terms of the amount of operator time required per unit of sequence data.

Nucleic acid sequence information may be obtained during the course of a single sequencing run, using a single nucleic acid molecule. Alternatively, multiple copies of a nucleic acid molecule may be sequenced in parallel or sequentially to confirm the nucleic acid sequence or to obtain complete sequence data. In other alternatives, both the nucleic acid molecule and its complementary strand may be sequenced to confirm the accuracy of the sequence information. Nucleotides may be released from a surface-attached nucleic acid, for example by exonuclease treatment. Released nucleotides may be transported, for example, through a microfluidic system to a Raman detector, to allow detection of released nucleotides without background Raman signals from the nucleic acid, exonuclease and/or other components of the system. Although certain methods disclosed herein involve nucleic acid sequencing, the skilled artisan will realize that the same type of methods may be utilized to obtain other information about nucleic acids, such as the form(s) of one or more single-nucleotide polymorphisms (SNPs) or other genetic variations present in a sample.

In certain embodiments of the invention, the nucleic acid to be sequenced is DNA, although it is contemplated that other nucleic acids comprising RNA or synthetic nucleotide analogs could be sequenced as well. The following detailed description contains numerous specific details in order to provide a more thorough understanding of the disclosed methods and apparatus. However, it will be apparent to those skilled in the art that the methods and apparatus may be practiced without these specific details. In other instances, devices, methods, procedures, and individual components that are well known in the art have not been described in detail herein.

In various embodiments of the invention, unlabeled nucleotides may be detected by Raman spectroscopy, for example by surface enhanced Raman spectroscopy (SERS), surface enhanced resonance Raman spectroscopy (SERRS), coherent anti-Stokes Raman spectroscopy (CARS) or other known Raman detection techniques. Alternatively, nucleotides may be covalently attached to Raman labels to enhance the Raman signal. In some embodiments, labeled nucleotides may be incorporated into a newly synthesized nucleic acid strand using standard nucleic acid polymerization techniques. Typically, either a primer of specific sequence or one or more random primers is allowed to hybridize to a template nucleic acid. Upon addition of a polymerase and labeled nucleotides, the Raman labeled nucleotides are covalently attached to the 3′ end of the primer, resulting in the formation of a labeled nucleic acid strand complementary in sequence to the template. The labeled strand may be separated from the unlabeled template, for example by heating to about 95° C. or other known methods. The two strands may be separated from each other by techniques well known in the art. For example, the primer oligonucleotide may be covalently modified with a biotin residue and the resulting biotinylated nucleic acid may be separated by binding to an avidin or streptavidin coated surface.

Either labeled or unlabeled single-stranded nucleic acid molecules may be digested with one or more exonucleases. The skilled artisan will realize that the disclosed methods are not limited to exonucleases per se, but may utilize any enzyme or other reagent capable of sequentially removing nucleotides from at least one end of a nucleic acid. Labeled or unlabeled nucleotides may be sequentially released from the 3′ end of the nucleic acid. After separation from the nucleic acid, the nucleotides may be detected by a Raman detection unit. Information on sequentially detected nucleotides may be used to compile a sequence of the nucleic acid. Nucleotides released from the 3′ end of a nucleic acid may be transported down a microfluidic flow path past a Raman detector. The detector may be capable of detecting labeled or unlabeled nucleotides at the single molecule level. The order of detection of the nucleotides by the Raman detector is the same as the order in which the nucleotides are released from the 3′ end of the nucleic acid. The sequence of the nucleic acid can thus be determined by the order in which released nucleotides are detected. Where a complementary strand is sequenced, the template strand will be complementary in sequence according to standard Watson-Crick hydrogen bond base-pairing (i.e., adenosine “A” to thymidine “T” and guanosine “G” to cytidine “C”).

In certain alternative methods, a tag molecule may be added to a reaction chamber or flow path upstream of the detection unit. The tag molecule binds to and tags free nucleotides as they are released from the nucleic acid molecule. This post-release tagging avoids problems that are encountered when the nucleotides of the nucleic acid molecule are tagged before their release into solution. For example, the use of bulky Raman label molecules may provide steric hindrance when each nucleotide incorporated into a nucleic acid molecule is labeled before exonuclease treatment, reducing the efficiency and increasing the time required for the sequencing reaction.

In certain embodiments of the invention, each of the four types of nucleotide may be attached to a distinguishable Raman label. Other alternatives are available, such as only incorporating Raman labels into pyrimidine residues (C and T). By labeling only pyrimidines and sequencing both strands of double-stranded DNA, the complete sequence of the DNA molecule may be obtained. Each nucleotide in a single-stranded DNA molecule must be either a purine or a pyrimidine. Where the nucleotide is a purine, it must be hydrogen bonded to a pyrimidine in the complementary strand. Thus, by sequencing all pyrimidines in both strands, the complete sequence is obtained. In one exemplary embodiment, the labeled nucleotides may comprise biotin-labeled deoxycytidine-5′-triphosphate (biotin-dCTP) and digoxigenin-labeled deoxyuridine-5′-triphosphate (digoxigenin-dUTP).

In alternative methods, no nucleotides are labeled and the unlabeled nucleotides are identified by Raman spectroscopy. As discussed above, it is possible to only identify half of the nucleotides and obtain complete sequence data by sequencing both strands of double-stranded DNA. For example, only adenosine and guanosine nucleotides may be identified and both strands may be sequenced, resulting in complete sequence determination.

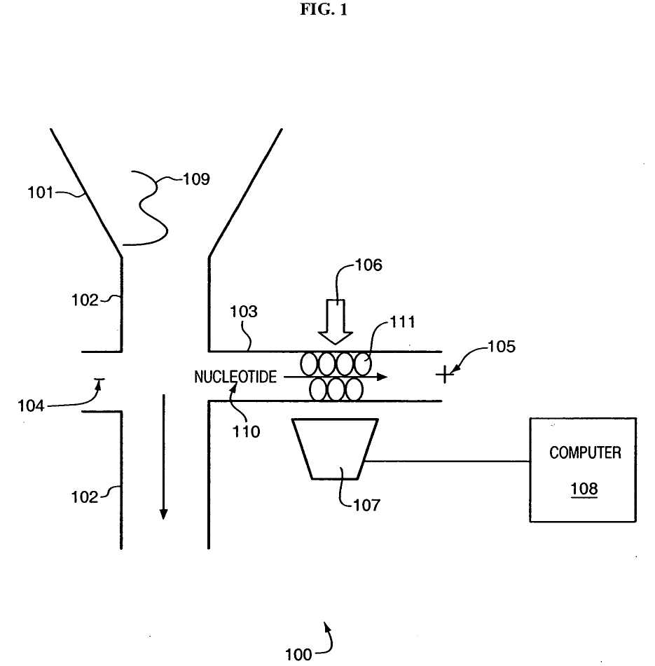

In various embodiments of the invention, exemplified in FIG. 1, nucleotides 110 are sequentially removed from one or more nucleic acid molecules 109, for example by treatment with exonuclease. The nucleotides 110 exit from a reaction chamber 101 and pass into a microfluidic channel 102. The microfluidic channel 102 is in fluid communication with a channel 103, which may be a nanochannel or microchannel. The nucleotides 110 may enter the nanochannel 103 or microchannel 103 in response to an electric field, negative on the microfluidic channel 102 side and positive on the nanochannel 103 or microchannel 103 side. The electric field may be imposed, for example, through the use of negative 104 and positive 105 electrodes. As nucleotides 110 pass down the nanochannel 103 or microchannel 103, they may pass through a region of closely packed nanoparticles 111. The nanoparticles 111 may be treated to form “hot spots”. Nucleotides 110 associated with a “hot spot” produce an enhanced Raman signal that may be detected using a detection unit comprising, for example, a laser 106 and CCD camera 107. Raman signals detected by the CCD camera 107 may be processed by an attached computer 108. The identity and time of passage of each nucleotide 110 through the nanoparticles 111 may be recorded and used to construct the sequence of the nucleic acid 109. In some embodiments of the invention, the nucleotides 110 are unmodified. In alternative embodiments of the invention, the nucleotides 110 may be covalently modified, for example by attachment of Raman labels.

Definitions

As used herein, “a” or “an” may mean one or more than one of an item.

As used herein, a “multiplicity” of an item means two or more of the item.

As used herein, a “microchannel” is any channel with a cross-sectional diameter of between 1 micrometer (μm) and 999 μm, while a “nanochannel” is any channel with a cross-sectional diameter of between 1 nanometer (nm) and 999 nm. In certain embodiments of the invention, a “nanochannel or microchannel” may be about 1 μm or less in diameter. A “microfluidic channel” is a channel in which liquids may move by microfluidic flow. The effects of channel diameter, fluid viscosity and flow rate on microfluidic flow are known in the art.

As used herein, “operably coupled” means that there is a functional interaction between two or more units. For example, a Raman detector may be “operably coupled” to a nanochannel or microchannel if the detector is arranged so that it can detect analytes, such as nucleotides, as they pass through the nanochannel or microchannel.

“Nucleic acid” encompasses DNA, RNA, single-stranded, double-stranded or triple stranded and any chemical modifications thereof. Virtually any modification of the nucleic acid is contemplated. A “nucleic acid” may be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100, 000, 150, 000, 200, 000, 500,000, 1,000,000, 1,500,000, 2,000,000, 5,000,000 or even more bases in length, up to a full-length chromosomal DNA molecule.

A “nucleoside” is a molecule comprising a purine or pyrimidine base or any chemical modification or structural analog thereof, covalently attached to a pentose sugar such as deoxyribose or ribose or derivatives or analogs of pentose sugars.

A “nucleotide” refers to a nucleoside further comprising at least one phosphate group covalently attached to the pentose sugar. The nucleotides to be detected may be ribonucleoside monophosphates or deoxyribonucleoside monophosphates although nucleoside diphosphates or triphosphates might be used. Alternatively, nucleosides may be released from the nucleic acid and detected. In other alternatives, purines or pyrimidines may be released, for example by acid treatment, and detected by Raman spectroscopy. Various substitutions or modifications may be made in the structure of the nucleotides, so long as they are still capable of being released from the nucleic acid, for example by exonuclease activity. For example, the ribose or deoxyribose moiety may be substituted with another pentose sugar or a pentose sugar analog. The phosphate groups may be substituted by various analogs. The purine or pyrimidine bases may be substituted or covalently modified. In embodiments involving labeled nucleotides, the label may be attached to any portion of the nucleotide so long as it does not interfere with exonuclease treatment.

A “Raman label” may be any organic or inorganic molecule, atom, complex or structure capable of producing a detectable Raman signal, including but not limited to synthetic molecules, dyes, naturally occurring pigments such as phycoerythrin, organic nanostructures such as C₆₀, buckyballs and carbon nanotubes, metal nanostructures such as gold or silver nanoparticles or nanoprisms and nano-scale semiconductors such as quantum dots. Numerous examples of Raman labels are disclosed below. The skilled artisan will realize that such examples are not limiting, and that “Raman label” encompasses any organic or inorganic atom, molecule, compound or structure known in the art that can be detected by Raman spectroscopy.

Nanoparticles

Certain embodiments of the invention involve the use of nanoparticles to enhance the Raman signal obtained from nucleotides. The nanoparticles may be silver or gold nanoparticles, although any nanoparticles capable of providing a surface enhanced Raman spectroscopy (SERS), surface enhanced resonance Raman spectroscopy (SERRS) and/or coherent anti-Stokes Raman spectroscopy (CARS) signal may be used. Nanoparticles of between 1 nm and 2 μm in diameter may be used. Alternatively, nanoparticles of 2 nm to 1 μm, 5 nm to 500 nm, 10 nm to 200 nm, 20 nm to 100 nm, 30 nm to 80 nm, 40 nm to 70 nm or 50 nm to 60 nm diameter may be used. Nanoparticles with an average diameter of 10 to 50 nm, 50 to 100 nm or about 100 nm are contemplated for certain applications. The nanoparticles may be approximately spherical in shape, although nanoparticles of any shape or of irregular shape may be used. Methods of preparing nanoparticles are known (e.g., U.S. Pat. Nos. 6,054,495; 6,127,120; 6,149,868; Lee and Meisel, J. Phys. Chem. 86:3391-3395, 1982). Nanoparticles may also be commercially obtained (e.g., Nanoprobes Inc., Yaphank, N.Y.; Polysciences, Inc., Warrington, Pa.; Ted-pella Inc., Redding, Calif.).

In certain embodiments of the invention, the nanoparticles may be random aggregates of nanoparticles (colloidal nanoparticles). In other embodiments, nanoparticles may be cross-linked to produce particular aggregates of nanoparticles, such as dimers, trimers, tetramers or other aggregates. Formation of “hot spots” for SERS, SERRS and/or CARS detection may be associated with particular aggregates of nanoparticles. Certain alternative embodiments may use heterogeneous mixtures of aggregates of different size or homogenous populations of nanoparticle aggregates. Aggregates containing a selected number of nanoparticles (dimers, trimers, etc.) may be enriched or purified by known techniques, such as ultracentrifugation in sucrose solutions. Nanoparticle aggregates of about 100, 200, 300, 400, 500, 600, 700, 800, 900 to 1000 nm in size or larger are contemplated. Nanoparticle aggregates may be between about 100 nm and about 200 nm in size.

Methods of cross-linking nanoparticles are known in the art (see, e.g., Feldheim, “Assembly of metal nanoparticle arrays using molecular bridges,” The Electrochemical Society Interface, Fall, 2001, pp. 22-25). Reaction of gold nanoparticles with linker compounds bearing terminal thiol or sulfhydryl groups is known (Feldheim, 2001). A single linker compound may be derivatized with thiol groups at both ends. Upon reaction with gold nanoparticles, the linker may form nanoparticle dimers that are separated by the length of the linker. Linkers with three, four or more thiol groups may be used to simultaneously attach to multiple nanoparticles (Feldheim, 2001). The use of an excess of nanoparticles to linker compounds prevents formation of multiple cross-links and nanoparticle precipitation. Aggregates of silver nanoparticles may be formed by standard synthesis methods known in the art.

Alternatively, the linker compounds used may contain a single reactive group, such as a thiol group. Nanoparticles containing a single attached linker compound may self-aggregate into dimers, for example, by non-covalent interaction of linker compounds attached to two different nanoparticles. For example, the linker compound may comprise alkane thiols. Following attachment of the thiol group to gold nanoparticles, the alkane groups will tend to associate by hydrophobic interaction. In other alternatives, the linker compounds may contain different functional groups at either end. For example, a linker compound could contain a sulfhydryl group at one end to allow attachment to gold nanoparticles, and a different reactive group at the other end to allow attachment to other linker compounds. Many such reactive groups are known in the art and may be used in the present methods and apparatus.

Gold or silver nanoparticles may be coated with derivatized silanes, such as aminosilane, 3-glycidoxypropyltrimethoxysilane (GOP) or aminopropyltrimethoxysilane (APTS). The reactive groups at the ends of the silanes may be used to form cross-linked aggregates of nanoparticles. It is contemplated that the linker compounds used may be of almost any length, ranging from about 0.05, 0.1, 0.2, 0.5, 0.75, 1, 2, 3, 4, 5, 6, 7, 8, 9, 10, 11, 12, 13, 14, 15, 16, 17, 18, 19, 20, 21, 22, 23, 24, 25, 27, 30, 35, 40, 45, 50, 55, 60, 65, 70, 80, 90 to 100 nm or even greater length. Linkers of heterogeneous length may be used.

The nanoparticles may be modified to contain various reactive groups before they are attached to linker compounds. Modified nanoparticles are commercially available, such as the Nanogold® nanoparticles from Nanoprobes, Inc. (Yaphank, N.Y.). Nanogold® nanoparticles may be obtained with either single or multiple maleimide, amine or other groups attached per nanoparticle. The Nanogold® nanoparticles are also available in either positively or negatively charged form to facilitate manipulation of nanoparticles in an electric field. Such modified nanoparticles may be attached to a variety of known linker compounds to provide dimers, trimers or other aggregates of nanoparticles.

The type of linker compound used is not limiting, so long as it results in the production of small aggregates of nanoparticles that will not precipitate in solution. The linker group may comprise phenylacetylene polymers (Feldheim, 2001). Alternatively, linker groups may comprise polytetrafluoroethylene, polyvinyl pyrrolidone, polystyrene, polypropylene, polyacrylamide, polyethylene or other known polymers. The linker compounds of use are not limited to polymers, but may also include other types of molecules such as silanes, alkanes, derivatized silanes or derivatized alkanes. Linker compounds of relatively simple chemical structure, such as alkanes or silanes, may be used to avoid interfering with the Raman signals emitted by nucleotides.

Where nanoparticles are packed into a nanochannel or microchannel, the nanoparticle aggregates may be manipulated into the channel by any method known in the art, such as microfluidics or nanofluidics, hydrodynamic focusing or electro-osmosis. Charged linker compounds or charged nanoparticles may be used to facilitate packing of nanoparticles into a channel through the use of electrical gradients.

Channels, Reaction Chambers and Integrated Chips

Materials

A reaction chamber, microfluidic channel, nanochannel or microchannel and other components of an apparatus may be formed as a single unit, for example in the form of a chip as known in semiconductor chips and/or microcapillary or microfluidic chips. Any materials known for use in such chips may be used in the disclosed apparatus, including silicon, silicon dioxide, silicon nitride, polydimethyl siloxane (PDMS), polymethylmethacrylate (PMMA), plastic, glass, quartz, etc. Part or all of the apparatus may be selected to be transparent to electromagnetic radiation at the excitation and emission frequencies used for Raman spectroscopy, such as glass, silicon, quartz or any other optically clear material. For fluid-filled compartments that may be exposed to nucleic acids and/or nucleotides, such as the reaction chamber, microfluidic channel and nanochannel or microchannel, the surfaces exposed to such molecules may be modified by coating, for example to transform a surface from a hydrophobic to a hydrophilic surface and/or to decrease adsorption of molecules to a surface. Surface modification of common chip materials such as glass, silicon and/or quartz is known in the art (e.g., U.S. Pat. No. 6,263,286). Such modifications may include, but are not limited to, coating with commercially available capillary coatings (Supelco, Bellafonte, Pa.), silanes with various functional groups such as polyethyleneoxide or acrylamide, or any other coating known in the art.

Integrated Chip Manufacture

Techniques for batch fabrication of chips are well known in the fields of computer chip manufacture and/or microcapillary chip manufacture. Such chips may be manufactured by any method known in the art, such as by photolithography and etching, laser ablation, injection molding, casting, molecular beam epitaxy, dip-pen nanolithography, chemical vapor deposition (CVD) fabrication, electron beam or focused ion beam technology or imprinting techniques. Non-limiting examples include conventional molding with a flowable, optically clear material such as plastic or glass; photolithography and dry etching of silicon dioxide; electron beam lithography using polymethylmethacrylate resist to pattern an aluminum mask on a silicon dioxide substrate, followed by reactive ion etching. Microfluidic channels may be made by molding polydimethylsiloxane (PDMS) according to Anderson et al. (“Fabrication of topologically complex three-dimensional microfluidic systems in PDMS by rapid prototyping,” Anal. Chem. 72:3158-3164, 2000). Methods for manufacture of nanoelectromechanical systems may be used. (See, e.g., Craighead, Science 290:1532-36, 2000.) Microfabricated chips are commercially available from sources such as Caliper Technologies Inc. (Mountain View, Calif.) and ACLARA BioSciences Inc. (Mountain View, Calif.).

Microfluidic Channels and Microchannels

Nucleotides released from one or more nucleic acid molecules may be moved down a microfluidic channel and then into a channel, which may be a nanochannel or microchannel. In certain embodiments, a microchannel or nanochannel may have a diameter between about 3 nm and about 1 μm. The diameter of the channel may be selected to be slightly smaller in size than an excitatory laser beam. The microfluidic channel and/or channel may comprise a microcapillary (available, e.g., from ACLARA BioSciences Inc., Mountain View, Calif.) or a liquid integrated circuit (e.g., Caliper Technologies Inc., Mountain View, Calif.). Such microfluidic platforms require only nanoliter volumes of sample. Nucleotides may move down a microfluidic channel by bulk flow of solvent, by electro-osmosis or by any other technique known in the art.

Alternatively, microcapillary electrophoresis may be used to transport nucleotides. Microcapillary electrophoresis generally involves the use of a thin capillary or channel that may or may not be filled with a particular separation medium. Electrophoresis of appropriately charged molecular species, such as negatively charged nucleotides, occurs in response to an imposed electrical field. Although electrophoresis is often used for size separation of a mixture of components that are simultaneously added to a microcapillary, it can also be used to transport similarly sized nucleotides that are sequentially released from a nucleic acid molecule. Because the purine nucleotides are larger than the pyrimidine nucleotides and would therefore migrate more slowly, the length of the various channels and corresponding transit time past the detector may be kept to a minimum to prevent differential migration from mixing up the order of nucleotides released from the nucleic acid. Alternatively, the separation medium filling the microcapillary may be selected so that the migration rates of purine and pyrimidine nucleotides are similar or identical. Methods of microcapillary electrophoresis have been disclosed, for example, by Woolley and Mathies (Proc. Natl. Acad. Sci. USA 91:11348-352, 1994).

Microfabrication of microfluidic devices, including microcapillary electrophoretic devices has been discussed in, e.g., Jacobsen et al. (Anal. Biochem, 209:278-283,1994); Effenhauser et al. (Anal. Chem. 66:2949-2953, 1994); Harrison et al. (Science 261:895-897, 1993) and U.S. Pat. No. 5,904,824. Typically, these methods comprise photolithographic etching of micron scale channels on silica, silicon or other crystalline substrates or chips, and can be readily adapted for use in the disclosed methods and apparatus. Smaller diameter channels, such as nanochannels, may be prepared by known methods, such as coating the inside of a microchannel to narrow the diameter, or using nanolithography, focused electron beam, focused ion beam or focused atom laser techniques. To facilitate detection of nucleotides, the material comprising the nanochannel or microchannel may be selected to be transparent to electromagnetic radiation at the excitation and emission frequencies used. Glass, silicon, and any other materials that are generally transparent in the frequency ranges used for Raman spectroscopy may be used. The nanochannel or microchannel may be fabricated from the same materials used for fabrication of the reaction chamber using injection molding or other known techniques.

Nanochannels

Fabrication of nanochannels may utilize any technique known in the art for nanoscale manufacturing. The following techniques are exemplary only. Nanochannels may be made, for example, using a high-throughput electron-beam lithography system. Electron beam lithography may be used to write features as small as 5 nm on silicon chips. Sensitive resists, such as polymethyl-methacrylate, coated on silicon surfaces may be patterned without use of a mask. The electron beam array may combine a field emitter cluster with a microchannel amplifier to increase the stability of the electron beam, allowing operation at low currents. The SoftMask™ computer control system may be used to control electron beam lithography of nanoscale features on a silicon or other chip.

Alternatively, nanochannels may be produced using focused atom lasers. (e.g., Bloch et al., “Optics with an atom laser beam,” Phys. Rev. Lett. 87:123-321, 2001.) Focused atom lasers may be used for lithography, much like standard lasers or focused electron beams. Such techniques are capable of producing micron scale or even nanoscale structures on a chip. Dip-pen nanolithography may also be used to form nanochannels. (e.g., Ivanisevic et al., “‘Dip-Pen’ Nanolithography on Semiconductor Surfaces,” J. Am. Chem. Soc., 123: 7887-7889, 2001.) Dip-pen nanolithography uses atomic force microscopy to deposit molecules on surfaces, such as silicon chips. Features as small as 15 nm in size may be formed, with spatial resolution of 10 nm. Nanoscale channels may be formed by using dip-pen nanolithography in combination with regular photolithography techniques. For example, a micron scale line in a layer of resist may be formed by standard photolithography. Using dip-pen nanolithography, the width of the line (and the corresponding diameter of the channel after etching) may be narrowed by depositing additional resist compound on the edges of the resist. After etching of the thinner line, a nanoscale channel may be formed. Alternatively, atomic force microscopy may be used to remove photoresist to form nanometer scale features.

Ion-beam lithography may also be used to create nanochannels on a chip. (e.g., Siegel, “Ion Beam Lithography,” VLSI Electronics, Microstructure Science, Vol. 16, Einspruch and Watts eds., Academic Press, New York, 1987.) A finely focused ion beam may be used to directly write features, such as nanochannels, on a layer of resist without use of a mask. Alternatively, broad ion beams may be used in combination with masks to form features as small as 100 nm in scale. Chemical etching, for example with hydrofluoric acid, may be used to remove exposed silicon that is not protected by resist. The skilled artisan will realize that the techniques disclosed above are not limiting, and that nanochannels may be formed by any method known in the art.

Reaction Chamber

The reaction chamber may be designed to hold the nucleic acid molecule and exonuclease in an aqueous environment. The reaction chamber may also hold an immobilization surface to which nucleic acid molecules may be attached. The reaction chamber may be designed to be temperature controlled, for example by incorporation of Pelletier elements or other known methods. A variety of methods of controlling temperature for low volume liquids are known in the art. (See, e.g., U.S. Pat. Nos. 5,038,853, 5,919,622, 6,054,263 and 6,180,372.) The reaction chamber may have an internal volume of about 1, 2, 5, 10, 20, 50, 100, 250, 500 or 750 picoliters, about 1, 2, 5, 10, 20, 50, 100, 250, 500 or 750 nanoliters, about 1, 2, 5, 10, 20, 50, 100, 250, 500 or 750 microliters, or about 1 milliliter. Reaction chambers may be manufactured using known chip technologies as discussed above.

Nucleic Acids

Nucleic acid molecules to be sequenced may be prepared by any technique known in the art. For example, the nucleic acids may be naturally occurring DNA or RNA molecules. Virtually any naturally occurring nucleic acid may be prepared and sequenced by the disclosed methods including, without limit, chromosomal, mitochondrial and chloroplast DNA and ribosomal, transfer, heterogeneous nuclear and messenger RNA. Methods for preparing and isolating various forms of cellular nucleic acids are known. (See, e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989). The methods disclosed in the cited references are exemplary only and any variation known in the art may be used. In cases where single stranded DNA (ssDNA) is to be sequenced, an ssDNA may be prepared from double stranded DNA (dsDNA) by any known method. Such methods may involve heating dsDNA and allowing the strands to separate, or may alternatively involve preparation of ssDNA from dsDNA by known amplification or replication methods, such as cloning into M13. Any such known method may be used to prepare ssDNA or ssRNA.

Virtually any type of nucleic acid that can serve as a substrate for an exonuclease or the equivalent may be used. For example, nucleic acids prepared by various amplification techniques, such as polymerase chain reaction (PCR™) amplification, may be sequenced. (See U.S. Pat. Nos. 4,683,195, 4,683,202 and 4,800,159.) Nucleic acids to be sequenced may alternatively be cloned in standard vectors, such as plasmids, cosmids, BACs (bacterial artificial chromosomes) or YACs (yeast artificial chromosomes). (See, e.g., Berger and Kimmel, 1987; Sambrook et al., 1989.) Nucleic acid inserts may be isolated from vector DNA, for example, by excision with appropriate restriction endonucleases, followed by agarose gel electrophoresis. Methods for isolation of insert nucleic acids are known in the art.

Isolation of Single Nucleic Acid Molecules

The nucleic acid molecule to be sequenced may be a single molecule of ssDNA or ssRNA. A variety of methods for selection and manipulation of single ssDNA or ssRNA molecules may be used, for example, hydrodynamic focusing, micro-manipulator coupling, optical trapping, or a combination of these and similar methods. (See, e.g., Goodwin et al., 1996, Acc. Chem. Res. 29:607-619; U.S. Pat. Nos. 4,962,037; 5,405,747; 5,776,674; 6,136,543; 6,225,068.)

Microfluidics or nanofluidics may be used to sort and isolate nucleic acid molecules. Hydrodynamics may be used to manipulate nucleic acids into a microchannel, microcapillary, or a micropore. Hydrodynamic forces may be used to move nucleic acid molecules across a comb structure to separate single nucleic acid molecules. Once the nucleic acid molecules have been separated, hydrodynamic focusing may be used to position the molecules within a reaction chamber. A thermal or electric potential, pressure or vacuum may also be used to provide a motive force for manipulation of nucleic acids. Manipulation of nucleic acids for sequencing may involve the use of a channel block design incorporating microfabricated channels and an integrated gel material, as disclosed in U.S. Pat. Nos. 5,867,266 and 6,214,246.

A sample containing a nucleic acid molecule may be diluted prior to coupling to an immobilization surface. The immobilization surface may be in the form of magnetic or non-magnetic beads or other discrete structural units. At an appropriate dilution, each bead will have a statistical probability of binding zero or one nucleic acid molecule. Beads with one attached nucleic acid molecule may be identified using, for example, fluorescent dyes and flow cytometer sorting or magnetic sorting. Depending on the relative sizes and uniformity of the beads and the nucleic acids, it may be possible to use a magnetic filter and mass separation to separate beads containing a single bound nucleic acid molecule. Alternatively, multiple nucleic acids attached to a single bead or other immobilization surface may be sequenced.

A coated fiber tip may also be used to generate single molecule nucleic acids for sequencing (e.g., U.S. Pat. No. 6,225,068). An immobilization surface may be prepared to contain a single molecule of avidin or other cross-linking agent. Such a surface may attach a single biotinylated nucleic acid molecule to be sequenced. This method not limited to the avidin-biotin binding system, but may be adapted to any coupling system known in the art.

In other alternatives, an optical trap may be used for manipulation of single molecule nucleic acid molecules for sequencing. (E.g., U.S. Pat. No. 5,776,674). Exemplary optical trapping systems are commercially available from Cell Robotics, Inc. (Albuquerque, N. Mex.), S+L GmbH (Heidelberg, Germany) and P.A.L.M. Gmbh (Wolfratshausen, Germany).

Methods of Immobilization

In various embodiments of the invention, the nucleic acid molecules to be sequenced may be attached to a solid surface (immobilized). Immobilization of nucleic acid molecules may be achieved by a variety of methods involving either non-covalent or covalent attachment between the nucleic acid molecule and the surface. In an exemplary embodiment, immobilization may be achieved by coating a surface with streptavidin or avidin and attachment of a biotinylated nucleic acid (Holmstrom et al., Anal. Biochem. 209:278-283, 1993). Immobilization may also occur by coating a silicon, glass or other surface with poly-L-Lys (lysine) or poly L-Lys, Phe (phenylalanine), followed by covalent attachment of either amino- or sulfhydryl-modified nucleic acids using bifunctional crosslinking reagents (Running et al., BioTechniques 8:276-277, 1990; Newton et al., Nucleic Acids Res. 21:1155-62, 1993). Amine residues may be introduced onto a surface through the use of aminosilane for cross-linking.

Immobilization may take place by direct covalent attachment of 5′-phosphorylated nucleic acids to chemically modified surfaces (Rasmussen et al., Anal. Biochem. 198:138-142, 1991). The covalent bond between the nucleic acid and the surface is formed by condensation with a water-soluble carbodiimide. This method facilitates a predominantly 5′-attachment of the nucleic acids via their 5′-phosphates.

DNA is commonly bound to glass by first silanizing the glass surface, then activating with carbodiimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule. DNA may be bound directly to membrane surfaces using ultraviolet radiation. Other non-limiting examples of immobilization techniques for nucleic acids are disclosed in U.S. Pat. Nos. 5,610,287, 5,776,674 and 6,225,068.

The type of surface to be used for immobilization of the nucleic acid is not limiting. The immobilization surface may be magnetic beads, non-magnetic beads, a planar surface, a pointed surface, or any other conformation of solid surface comprising almost any material, so long as the material is sufficiently durable and inert to allow the nucleic acid sequencing reaction to occur. Non-limiting examples of surfaces that may be used include glass, silica, silicate, PDMS, silver or other metal coated surfaces, nitrocellulose, nylon, activated quartz, activated glass, polyvinylidene difluoride (PVDF), polystyrene, polyacrylamide, other polymers such as poly(vinyl chloride), poly(methyl methacrylate) or poly(dimethyl siloxane), and photopolymers which contain photoreactive species such as nitrenes, carbenes and ketyl radicals capable of forming covalent links with nucleic acid molecules (See U.S. Pat. Nos. 5,405,766 and 5,986,076).

Bifunctional cross-linking reagents may be used to attach a nucleic acid molecule to a surface. The bifunctional cross-linking reagents can be divided according to the specificity of their functional groups, e.g., amino, guanidino, indole, or carboxyl specific groups. Of these, reagents directed to free amino groups are popular because of their commercial availability, ease of synthesis and the mild reaction conditions under which they can be applied. Exemplary methods for cross-linking molecules are disclosed in U.S. Pat. Nos. 5,603,872 and 5,401,511. Cross-linking reagents include glutaraldehyde (GAD), bifunctional oxirane (OXR), ethylene glycol diglycidyl ether (EGDE), and carbodiimides, such as 1-ethyl-3-(3-dimethylaminopropyl) carbodiimide (EDC).

Nucleic Acid Synthesis

Polymerases

Certain methods disclosed herein may involve binding of a synthetic reagent, such as a DNA polymerase, to a primer molecule and the addition of Raman labeled nucleotides to the 3′ end of the primer. Non-limiting examples of polymerases include DNA polymerases, RNA polymerases, reverse transcriptases, and RNA-dependent RNA polymerases. The differences between these polymerases in terms of their “proofreading” activity and requirement or lack of requirement for primers and promoter sequences are known in the art. Where RNA polymerases are used as the polymerase, a template molecule to be sequenced may be double-stranded DNA. Non-limiting examples of polymerases include Thermatoga maritima DNA polymerase, AmplitaqFS™ DNA polymerase, Taquenase™ DNA polymerase, ThermoSequenase™, Taq DNA polymerase, Qbeta™ replicase, T4 DNA polymerase, Thermus thermophilus DNA polymerase, RNA-dependent RNA polymerase and SP6 RNA polymerase.

A number of polymerases are commercially available, including Pwo DNA Polymerase (Boehringer Mannheim Biochemicals, Indianapolis, Ind.); Bst Polymerase (Bio-Rad Laboratories, Hercules, Calif.); IsoTherm™ DNA Polymerase (Epicentre Technologies, Madison, Wis.); Moloney Murine Leukemia Virus Reverse Transcriptase, Pfu DNA Polymerase, Avian Myeloblastosis Virus Reverse Transcriptase, Thermus flavus (Tfl) DNA Polymerase and Thermococcus litoralis (Tli) DNA Polymerase (Promega Corp., Madison, Wis.); RAV2 Reverse Transcriptase, HIV-1 Reverse Transcriptase, T7 RNA Polymerase, T3 RNA Polymerase, SP6 RNA Polymerase, E. coli RNA Polymerase, Thermus aquaticus DNA Polymerase, T7 DNA Polymerase +/−3′→5′ exonuclease, Kienow Fragment of DNA Polymerase I, Thermus ‘ubiquitous’ DNA Polymerase, and DNA polymerase I (Amersham Pharmacia Biotech, Piscataway, N.J.). Any polymerase known in the art capable of template dependent polymerization of labeled nucleotides may be used. (See, e.g., Goodman and Tippin, Nat. Rev. Mol. Cell Biol. 1(2):101-9, 2000; U.S. Pat. No. 6,090,589.) Methods of using polymerases to synthesize nucleic acids from labeled nucleotides are known (e.g., U.S. Pat. Nos. 4,962,037; 5,405,747; 6,136,543; 6,210,896).

Primers

Generally, primers are between ten and twenty bases in length, although longer primers may be employed. Primers may be designed to be complementary in sequence to a known portion of a template nucleic acid molecule. Known primer sequences may be used, for example, where primers are selected for identifying sequence variants adjacent to known constant chromosomal sequences, where an unknown nucleic acid sequence is inserted into a vector of known sequence, or where a native nucleic acid has been partially sequenced. Methods for synthesis of primers of any sequence are known. Alternatively, random primers, such as random hexamers or random oligomers, may be used to initiate nucleic acid polymerization in the absence of a known primer-binding site.

Exonucleases

Methods of nucleic acid sequencing may involve binding of an exonuclease to the free end of a nucleic acid molecule and removal of nucleotides one at a time. The type of exonuclease that may be used is not limiting. Non-limiting examples of exonucleases of potential use include E. coli exonuclease I, III, V or VII, Bal 31 exonuclease, mung bean exonuclease, S1 nuclease, E. coli DNA polymerase I holoenzyme or Klenow fragment, RecJ, exonuclease T, T4 or T7 DNA polymerase, Taq polymerase, exonuclease T7 gene 6, snake venom phosphodiesterase, spleen phosphodiesterase, Thermococcus litoralis DNA polymerase, Pyrococcus sp. GB-D DNA polymerase, lambda exonuclease, S. aureus micrococcal nuclease, DNase I, ribonuclease A, T1 micrococcal nuclease, or other exonucleases known in the art. Exonucleases are available from commercial sources such as New England Biolabs (Beverly, Mass.), Amersham Pharmacia Biotech (Piscataway, N.J.), Promega (Madison, Wis.), Sigma Chemicals (St. Louis, Mo.) or Boehringer Mannheim (Indianapolis, Ind.).

The skilled artisan will realize that enzymes with exonuclease activity have various properties known in the art. The rate of exonuclease activity may be manipulated to coincide with the optimal rate of analysis of nucleotides by the detector. Various methods are known for adjusting the rate of exonuclease activity, including adjusting the temperature, pressure, pH, salt concentration or divalent cation concentration in the reaction chamber. Methods of optimization of exonuclease activity are known in the art.

Although nucleoside monophosphates will generally be released from nucleic acids by exonuclease activity, the disclosed methods are not limited to detection of any particular form of free nucleotide or nucleoside but encompass any monomer that may be released from a nucleic acid. In some cases, the molecule to be detected may be a purine or pyrimidine base that has been released from a nucleotide or nucleoside by acid hydrolysis, for example, as disclosed below.

Raman Labels

Certain methods disclosed herein may involve attaching a label to one or more nucleotides, nucleosides or bases to facilitate their detection by the Raman detector. Non-limiting examples of labels that may be used for Raman spectroscopy include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins and aminoacridine. These and other Raman labels may be obtained from commercial sources (e.g., Molecular Probes, Eugene, Oreg.).

Polycyclic aromatic compounds in general may function as Raman labels, as is known in the art. Other labels that may be of use include cyanide, thiol, chlorine, bromine, methyl, phosphorus and sulfur. Carbon nanotubes may also be of use as Raman labels. The use of labels in Raman spectroscopy is known (e.g., U.S. Pat. Nos. 5,306,403 and 6,174,677). The skilled artisan will realize that Raman labels should generate distinguishable Raman spectra when bound to different types of nucleotide.

Labels may be attached directly to the nucleotides or may be attached via various linker compounds. Alternatively, nucleotide precursors that are covalently attached to Raman labels are available from standard commercial sources (e.g., Roche Molecular Biochemicals, Indianapolis, Ind.; Promega Corp., Madison, Wis.; Ambion, Inc., Austin, Tex.; Amersham Pharmacia Biotech, Piscataway, N.J.). Raman labels that contain reactive groups designed to covalently react with other molecules, such as nucleotides, are commercially available (e.g., Molecular Probes, Eugene, Oreg.). Methods for preparing labeled nucleotides and incorporating them into nucleic acids are known (e.g., U.S. Pat. Nos. 4,962,037; 5,405,747; 6,136,543; 6,210,896).

Detection Unit

Exemplary apparatus disclosed herein may comprise a detection unit that is designed to detect and/or quantify nucleotides, nucleosides, purines and/or pyrimidines by Raman spectroscopy. Various methods for detection of nucleotides by Raman spectroscopy are known in the art. (See, e.g., U.S. Pat. Nos. 5,306,403; 6,002,471; 6,174,677). Such known methods typically involve detection of higher concentrations of nucleotides than may be identified by alternative known methods, such as fluorescence spectroscopy. Raman detection of nucleotides at the single molecule level has not been disclosed, prior to the present specification. Variations on surface enhanced Raman spectroscopy (SERS), surface enhanced resonance Raman spectroscopy (SERRS) and coherent anti-Stokes Raman spectroscopy (CARS) have been disclosed. In SERS and SERRS, the sensitivity of the Raman detection is enhanced by a factor of 106 or more for molecules adsorbed on roughened metal surfaces, such as silver, gold, platinum, copper or aluminum surfaces. A non-limiting example of a Raman detection unit is disclosed in U.S. Pat. No. 6,002,471.

An excitation beam may be generated by either an Nd:YAG laser at 532 nm wavelength or a Ti:sapphire laser at 365 nm wavelength. Pulsed laser beams or continuous laser beams may be used. An excitation beam may pass through confocal optics and a microscope objective, and may be focused onto a nanochannel or microchannel containing packed nanoparticles. The Raman emission light from the nucleotides may be collected by the microscope objective and confocal optics and coupled to a monochromator for spectral dissociation. The confocal optics may include a combination of dichroic filters, barrier filters, confocal pinholes, lenses, and mirrors for reducing the background signal. Standard full field optics may be used as well as confocal optics. The Raman emission signal may be detected by a Raman detector, which may include an avalanche photodiode interfaced with a computer for counting and digitization of the signal.

Alternative examples of detection units are disclosed, for example, in U.S. Pat. No. 5,306,403, including a Spex Model 1403 double-grating spectrophotometer equipped with a gallium-arsenide photomultiplier tube (RCA Model C31034 or Burle Industries Model C3103402) operated in the single-photon counting mode. The excitation source may comprise a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677). The excitation beam may be spectrally purified with a bandpass filter (Corion) and may be focused on a nanochannel or microchannel using a 6× objective lens (Newport, Model L6X). The objective lens may be used to both excite the nucleotides and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) may be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors may be used, such as charged injection devices, photodiode arrays or phototransistor arrays.

Any suitable form or configuration of Raman spectroscopy or related techniques known in the art may be used for detection of nucleotides, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

Information Processing and Control System and Data Analysis

A nucleic acid sequencing apparatus may comprise an information processing system. The type of information processing system used is not limiting. An exemplary information processing system may incorporate a computer comprising a bus for communicating information and a processor for processing information. The processor may be selected from the Pentium® family of processors, including without limitation the Pentium® II family, the Pentium® III family and the Pentium® 4 family of processors available from Intel Corp. (Santa Clara, Calif.). Alternatively, the processor may be a Celeron®, an Itanium®, or a Pentium Xeon® processor (Intel Corp., Santa Clara, Calif.). The processor may be based on Intel® architecture, such as Intel® IA-32 or Intel® IA-64 architecture. Alternatively, other processors may be used.

The detection unit may be operably coupled to the information processing system. Data from the detection unit may be processed by the processor and data stored in the main memory. Data on emission profiles for standard nucleotides may also be stored in main memory or in ROM. The processor may compare the emission spectra from nucleotides in the nanochannel or microchannel to identify the type of nucleotide released from the nucleic acid molecule. The main memory may also store the sequence of nucleotides released from the nucleic acid molecule. The processor may analyze the data from the detection unit to determine the sequence of the nucleic acid. Where only purines or pyrimidines are labeled and/or detected, the processor may compare the sequence of bases obtained from two complementary nucleic acid strands to generate the complete nucleic acid sequence.

While the processes described herein may be performed under the control of a programmed processor, the processes may also be fully or partially implemented by any programmable or hardcoded logic, such as Field Programmable Gate Arrays (FPGAs), TTL logic, or Application Specific Integrated Circuits (ASICs), for example. Additionally, the disclosed methods may be performed by any combination of programmed general purpose computer components and/or custom hardware components.

Following the data gathering operation, the data may be reported to a data analysis operation. To facilitate the analysis operation, the data obtained by the detection unit may be analyzed using a digital computer. The computer may be programmed for receipt and storage of the data from the detection unit as well as for analysis and reporting of the data gathered.

Custom designed software packages may be used to analyze the data obtained from the detection unit. Data analysis may also be performed using an information processing system and publicly available software packages. Non-limiting examples of available software for DNA sequence analysis include the PRISM™ DNA Sequencing Analysis Software (Applied Biosystems, Foster City, Calif.), the Sequencher™ package (Gene Codes, Ann Arbor, Mich.), and a variety of software packages available through the National Biotechnology Information Facility.

### EXAMPLES

### Example 1

### Nucleic Acid Sequencing Using Raman Detection and Nanoparticles

Certain embodiments of the invention, exemplified in FIG. 1, involve sequencing of one or more single-stranded nucleic acid molecules 109 that may be attached to an immobilization surface in a reaction chamber 101. The reaction chamber 101 may contain one or more exonucleases that sequentially remove one nucleotide 110 at a time from the unattached end of the nucleic acid molecule 109.

As the nucleotides 110 are released, they may move down a microfluidic channel 102 and into a nanochannel 103 or microchannel 103, past a detection unit. The detection unit may comprise an excitation source 106, such as a laser, that emits an excitatory beam. The excitatory beam may interact with the released nucleotides 110 so that electrons are excited to a higher energy state. The Raman emission spectrum that results from the return of the electrons to a lower energy state may be detected by a Raman spectroscopic detector 107, such as a spectrometer, a monochromator or a charge coupled device (CCD), such as a CCD camera.

The excitation source 106 and detector 107 may be arranged so that nucleotides 110 are excited and detected as they pass through a region of closely packed nanoparticles 111 in a nanochannel 103 or microchannel 103. The nanoparticles 111 may be cross-linked to form “hot spots” for Raman detection. By passing the nucleotides 110 through the nanoparticle 111 hot spots, the sensitivity of Raman detection may be increased by many orders of magnitude.

Preparation of Reaction Chamber, Microfluidic Channel and Microchannel

Borofloat glass wafers (Precision Glass & Optics, Santa Ana, Calif.) may be pre-etched for a short period in concentrated HF (hydrofluoric acid) and cleaned before deposition of an amorphous silicon sacrificial layer in a plasma-enhanced chemical vapor deposition (PECVD) system (PEII-A, Technics West, San Jose, Calif.). Wafers may be primed with hexamethyldisilazane (HMDS), spin-coated with photoresist (Shipley 1818, Marlborough, Mass.) and soft-baked. A contact mask aligner (Quintel Corp. San Jose, Calif.) may be used to expose the photoresist layer with one or more mask designs, and the exposed photoresist may be removed using a mixture of Microposit developer concentrate (Shipley) and water. Developed wafers may be hard-baked and the exposed amorphous silicon removed using CF₄ (carbon tetrafluoride) plasma in a PECVD reactor. Wafers may be chemically etched with concentrated HF to produce the reaction chamber 101, microfluidic channel 102 and microchannel 103. The remaining photoresist may be stripped and the amorphous silicon removed.

Nanochannels 103 may be formed by a variation of this protocol. Standard photolithography may be used to form the micron scale features of the integrated chip. A thin layer of resist may be coated onto the chip. An atomic force microscopy/scanning tunneling probe tip may be used to remove a 5 to 10 nm wide strip of resist from the chip surface. The chip may be briefly etched with dilute HF to produce a nanometer scale groove on the chip surface. In the present non-limiting example, a channel 103 with a diameter of between 500 nm and 1 μm may be prepared.

Access holes may be drilled into the etched wafers with a diamond drill bit (Crystalite, Westerville, Ohio). A finished chip may be prepared by thermally bonding two complementary etched and drilled plates to each other in a programmable vacuum furnace (Centurion VPM, J. M. Ney, Yucaipa, Calif.). Alternative exemplary methods for fabrication of a chip incorporating a reaction chamber 101, microfluidic channel 102 and nanochannel 103 or microchannel 103 are disclosed in U.S. Pat. Nos. 5,867,266 and 6,214,246. A nylon filter with a molecular weight cutoff of 2,500 daltons may be inserted between the reaction chamber 101 and the microfluidic channel 102 to prevent exonuclease and/or nucleic acid 109 from leaving the reaction chamber 101.

Nanoparticle Preparation

Silver nanoparticles 111 may be prepared according to Lee and Meisel (J. Phys. Chem. 86:3391-3395, 1982). Gold nanoparticles 111 may be purchased from Polysciences, Inc. (Warrington, Pa.), Nanoprobes, Inc. (Yaphank, N.Y.) or Ted-pella Inc. (Redding, Calif.). In a non-limiting example, 60 nm gold nanoparticles 111 may be used. The skilled artisan will realize that other sized nanoparticles 111, such as 5, 10, or 20 nm, may also be used.

Gold nanoparticles 111 may be reacted with alkane dithiols, with chain lengths ranging from 5 nm to 50 nm. The linker compounds may contain thiol groups at both ends of the alkane to react with gold nanoparticles 111. An excess of nanoparticles 111 to linker compounds may be used and the linker compounds slowly added to the nanoparticles 111 to avoid formation of large nanoparticle aggregates. After incubation for two hours at room temperature, nanoparticle 111 aggregates may be separated from single nanoparticles 111 by ultracentrifugation in 1 M sucrose. Electron microscopy reveals that aggregates prepared by this method contain from two to six nanoparticles 111 per aggregate. The aggregated nanoparticles 111 may be loaded into a microchannel 103 by microfluidic flow. A constriction or filter at the end of the microchannel 103 may be used to hold the nanoparticle aggregates 111 in place.

Nucleic Acid Preparation and Exonuclease Treatment

Human chromosomal DNA may be purified according to Sambrook et al. (1989). Following digestion with Bam HI, the genomic DNA fragments may be inserted into the multiple cloning site of the pBluescript® II phagemid vector (Stratagene, Inc., La Jolla, Calif.) and grown up in E. coli. After plating on ampicillin-containing agarose plates a single colony may be selected and grown up for sequencing. Single-stranded DNA copies of the genomic DNA insert may be rescued by co-infection with helper phage. After digestion in a solution of proteinase K:sodium dodecyl sulphate (SDS), the DNA may be phenol extracted and then precipitated by addition of sodium acetate (pH 6.5, about 0.3 M) and 0.8 volumes of 2-propanol. The DNA containing pellet may be resuspended in Tris-EDTA buffer and stored at −20° C. until use.

M13 forward primers complementary to the known pBluescript® sequence, located next to the genomic DNA insert, may be purchased from Midland Certified Reagent Company (Midland, Tex.). The primers may be covalently-modified to contain a biotin moiety attached to the 5′ end of the oligonucleotide. The biotin group may be covalently linked to the 5′-phosphate of the primer via a (CH₂)₆ spacer. Biotin-labeled primers may be allowed to hybridize to the ssDNA template molecules prepared from the pBluescript® vector. The primer-template complexes may be attached to streptavidine coated beads according to Dorre et al. (Bioimaging 5: 139-152, 1997). At appropriate DNA dilutions, a single primer-template complex is attached to a single bead. A bead containing a single primer-template complex may be inserted into the reaction chamber 101 of a sequencing apparatus 100.

The primer-template may be incubated with modified T7 DNA polymerase (United States Biochemical Corp., Cleveland, Ohio). The reaction mixture may contain unlabeled deoxyadenosine-5′-triphosphate (dATP) and deoxyguanosine-5′-triphosphate (dGTP), digoxigenin-labeled deoxyuridine-5′-triphosphate (digoxigenin-dUTP) and rhodamine-labeled deoxycytidine-5′-triphosphate (rhodamine-dCTP). The polymerization reaction may be allowed to proceed for 2 hours at 37° C. After synthesis of the digoxigenin and rhodamine labeled nucleic acid, the template strand may be separated from the labeled nucleic acid, and the template strand, DNA polymerase and unincorporated nucleotides washed out of the reaction chamber 101. Alternatively, all deoxynucleoside triphosphates used for polymerization may be unlabeled. In other alternatives, single stranded nucleic acids may be directly sequenced without polymerization of a complementary strand.

Exonuclease activity may be initiated by addition of exonuclease III to the reaction chamber 101. The reaction mixture may be maintained at pH 8.0 and 37° C. As nucleotides 110 are released from the 3′ end of the nucleic acid, they may be transported by microfluidic flow down the microfluidic channel 102. At the entrance to the microchannel 103, an electrical potential gradient created by a pair of electrodes 104, 105 may be used to drive the nucleotides 110 out of the microfluidic channel 102 and into the microchannel 103. As the nucleotides 110 pass through the packed nanoparticles 111, they may be exposed to excitatory radiation from a laser 106. Raman emission spectra may be detected by the Raman detector 107 as disclosed below.

Raman Detection of Nucleotides

A Raman detection unit as disclosed in Example 2 may be used. The Raman detector 107 may be capable of detecting and identifying single nucleotides 110 of DATP, dGTP, rhodamine-dCTP and digoxigenin-dUTP moving past the detector 107. Data on the time course for labeled nucleotide detection may be compiled and analyzed to obtain the sequence of the nucleic acid. In alternative embodiments, the detector 107 may be capable of detecting and identifying single unlabeled nucleotides.

### Example 2

### Raman Detection of Nucleotides

Methods and Apparatus

In a non-limiting example, the excitation beam of a Raman detection unit was generated by a titanium:sapphire laser (Mira by Coherent) at a near-infrared wavelength (750˜950 nm) or a gallium aluminum arsenide diode laser (PI-ECL series by Process Instruments) at 785 nm or 830 nm. Pulsed laser beams or continuous beams were used. The excitation beam was transmitted through a dichroic mirror (holographic notch filter by Kaiser Optical or a dichromatic interference filter by Chroma or Omega Optical) into a collinear geometry with the collected beam. The transmitted beam passed through a microscope objective (Nikon LU series), and was focused onto the Raman active substrate where target analytes (nucleotides or purine or pyrimidine bases) were located.

The Raman scattered light from the analytes was collected by the same microscope objective, and passed the dichroic mirror to the Raman detector. The Raman detector comprised a focusing lens, a spectrograph, and an array detector. The focusing lens focused the Raman scattered light through the entrance slit of the spectrograph. The spectrograph (Acton Research) comprised a grating that dispersed the light by its wavelength. The dispersed light was imaged onto an array detector (back-illuminated deep-depletion CCD camera by RoperScientific). The array detector was connected to a controller circuit, which was connected to a computer for data transfer and control of the detector function.

For surface-enhanced Raman spectroscopy (SERS), the Raman active substrate consisted of metallic nanoparticles or metal-coated nanostructures. Silver nanoparticles, ranging in size from 5 to 200 nm, was made by the method of Lee and Meisel (J. Phys. Chem., 86:3391, 1982). Alternatively, samples were placed on an aluminum substrate under the microscope objective. The Figures discussed below were collected in a stationary sample on the aluminum substrate. The number of molecules detected was determined by the optical collection volume of the illuminated sample.

Single nucleotides may also be detected by SERS using microfluidic channels. In various embodiments of the invention, nucleotides may be delivered to a Raman active substrate through a microfluidic channel (between about 5 and 200 μm wide). Microfluidic channels can be made by molding polydimethylsiloxane (PDMS), using the technique disclosed in Anderson et al. (“Fabrication of topologically complex three-dimensional microfluidic systems in PDMS by rapid prototyping,” Anal. Chem. 72:3158-3164, 2000).

Where SERS was performed in the presence of silver nanoparticles, the nucleotide, purine or pyrimidine analyte was mixed with LiCl (90 μM final concentration) and nanoparticles (0.25 M final concentration silver atoms). SERS data were collected using room temperature analyte solutions.

Results

Nucleoside monophosphates, purines and pyrimidines were analyzed by SERS, using the system disclosed above. Table I shows exemplary detection limits for various analytes of interest.

TABLE 1
SERS Detection of Nucleoside Monophosphates, Purines and Pyrimidines

| | | Number of Molecules |
|---|---|---|
| Analyte | Final Concentration | Detected |
| dAMP | 9 picomolar (pM) | ˜1 molecule |
| Adenine | 9 pM | ˜1 molecule |
| dGMP | 90 μM | 6 × 10⁶ |
| Guanine | 909 pM | 60 |
| dCMP | 909 μM | 6 × 10⁷ |
| Cyotosine | 90 nM | 6 × 10³ |
| dTMP | 9 μM | 6 × 10⁵ |
| Thymine | 90 nM | 6 × 10³ |

Conditions were optimized for adenine nucleotides only. LiCl (90 μM final concentration) was determined to provide optimal SERS detection of adenine nucleotides. Detection of other nucleotides may be facilitated by use of other alkali-metal halide salts, such as NaCl, KCl, RbCl or CsCl. The claimed methods are not limited by the electrolyte solution used, and it is contemplated that other types of electrolyte solutions, such as MgCl, CaCl, NaF, KBr, LiI, etc. may be of use. The skilled artisan will realize that electrolyte solutions that do not exhibit strong Raman signals will provide minimal interference with SERS detection of nucleotides. The results demonstrate that the Raman detection system and methods disclosed above were capable of detecting and identifying single molecules of nucleotides and purine bases. This is the first report of Raman detection of unlabeled nucleotides at the single nucleotide level.

### Example 3

### Raman Emission Spectra of Nucleotides, Purines and Pyrimidines

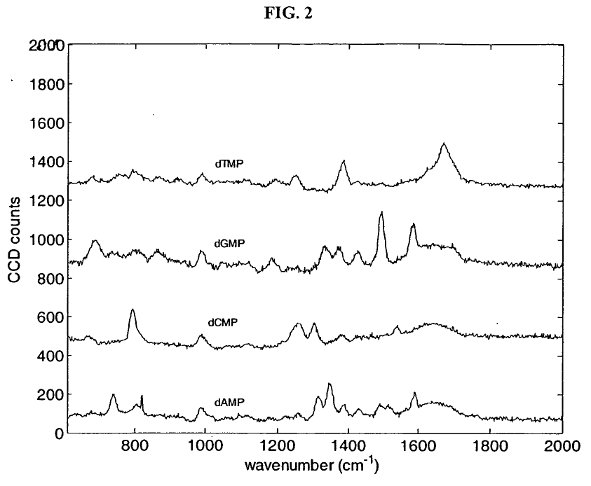

The Raman emission spectra of various analytes of interest was obtained using the protocol of Example 2, with the indicated modifications. FIG. 2 shows the Raman emission spectra of a 100 mM solution of each of the four nucleoside monophosphates, in the absence of surface enhancement and without Raman labels. No LiCi was added to the solution. A 10 second data collection time was used. Lower concentrations of nucleotides may be detected with longer collection times, with surface enhancement, using labeled nucleotides and/or with added electrolyte solution. Excitation occurred at 514 nm. For each of the following figures, a 785 nm excitation wavelength was used. As shown in FIG. 2, the unenhanced Raman spectra showed characteristic emission peaks for each of the four unlabeled nucleoside monophosphates.

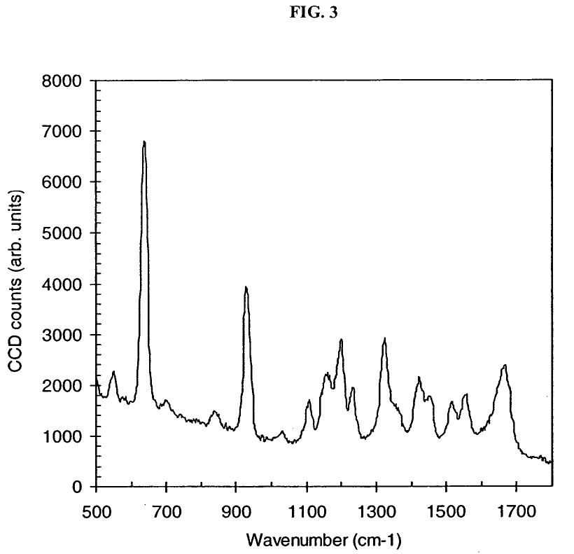

FIG. 3 shows the SERS spectrum of a 1 nm solution of guanine, in the presence of LiCl and silver nanoparticles. Guanine was obtained from dGMP by acid treatment, as discussed in Nucleic Acid Chemistry, Part 1, L. B. Townsend and R. S. Tipson (eds.), Wiley-Interscience, New York, 1978. The SERS spectrum was obtained using a 100 msec data collection time.

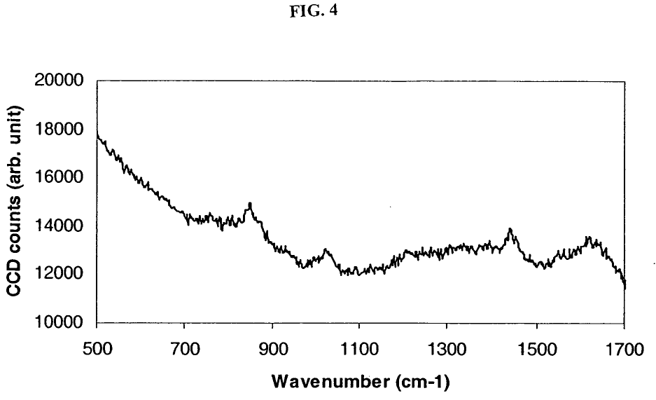

FIG. 4 shows the SERS spectrum of a 10 nM cytosine solution, obtained from dCMP by acid hydrolysis. Data were collected using a 1 second collection time.

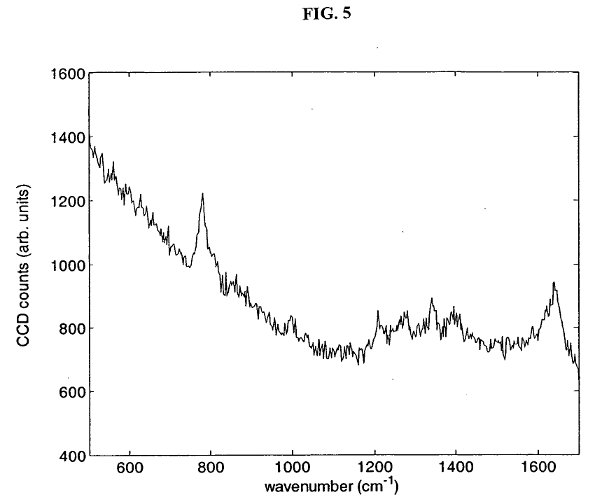

FIG. 5 shows the SERS spectrum of a 100 nM thymine solution, obtained by acid hydrolysis of dTMP. Data were collected using a 100 msec collection time.

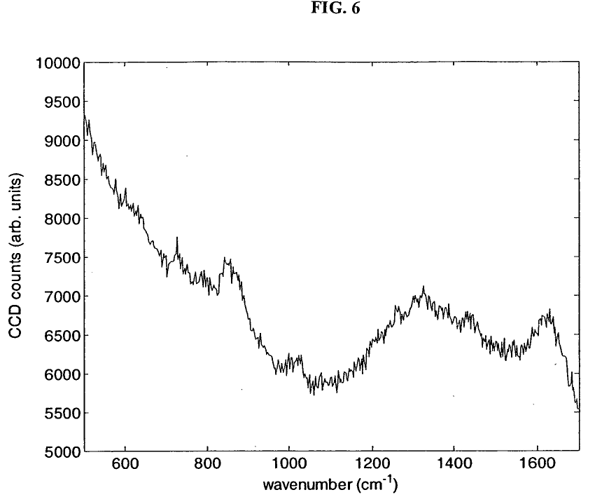

FIG. 6 shows the SERS spectrum of a 100 pM adenine solution, obtained by acid hydrolysis of dAMP. Data were collected for 1 second.

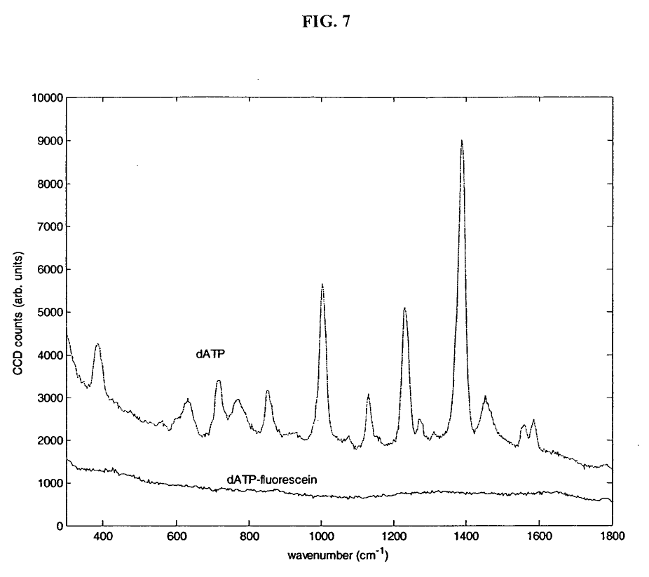

FIG. 7 shows the SERS spectrum of a 500 nM solution of dATP (lower trace) and fluorescein-labeled dATP (upper trace). dATP-fluorescein was purchased from Roche Applied Science (Indianapolis, Ind.). The Figure shows a strong increase in SERS signal due to labeling with fluorescein.

### Example 4

### SERS Detection of Nucleotides and Amplification Products

Silver Nanoparticle Formation

Silver nanoparticles used for SERS detection were produced according to Lee and Meisel (1982). Eighteen milligrams of AgNO₃ were dissolved in 100 mL (milliliters) of distilled water and heated to boiling. Ten mL of a 1% sodium citrate solution was added drop-wise to the AgNO₃ solution over a 10 min period. The solution was kept boiling for another hour. The resulting silver colloid solution was cooled and stored.

SERS Detection of Adenine

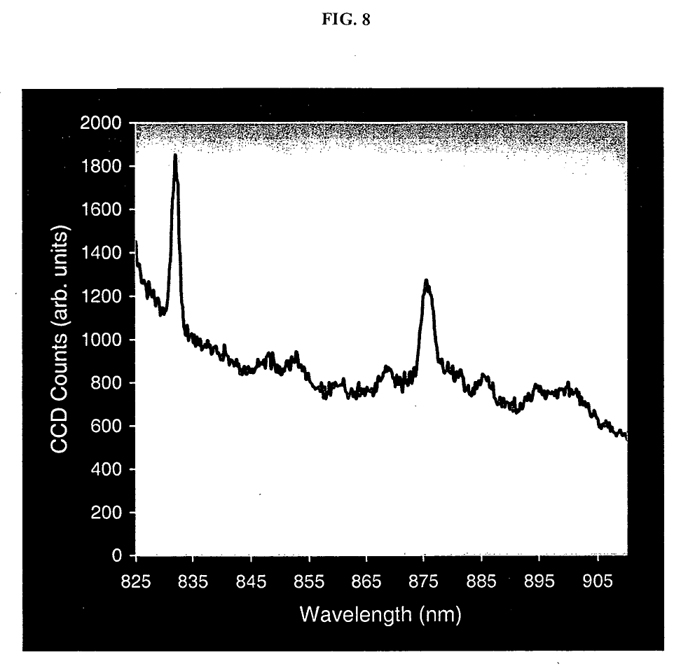

The Raman detection system was as disclosed in Example 2. One mL of silver colloid solution was diluted with 2 mL of distilled water. The diluted silver colloid solution (160 μL) (microliters) was mixed with 20 μL of a 10 nM (nanomolar) adenine solution and 40 μL of LiCl (0.5 molar) on an aluminum tray. The LiCl acted as a Raman enhancing agent for adenine. The final concentration of adenine in the sample was 0.9 nM, in a detection volume of about 100 to 150 femtoliters, containing an estimated 60 molecules of adenine. The Raman emission spectrum was collected using an excitation source at 785 nm excitation, with a 100 millisecond collection time. As shown in FIG. 8, this procedure showed the detection of 60 molecules of adenine, with strong emission peaks detected at about 833 nm and 877 nm. As discussed in Example 2, single molecule detection of adenine has been shown using the disclosed methods and apparatus.

Rolling Circle Amplification

One picomole (pmol) of a rolling circle amplification (RCA) primer was added to 0.1 pmol of circular, single-stranded M13 DNA template. The mixture was incubated with 1×T7 polymerase 160 buffer (20 mM (millimolar) Tris-HCl, pH 7.5, 10 mM MgCl2, 1 mM dithiothreitol), 0.5 mM dNTPs and 2.5 units of T7 DNA polymerase for 2 hours at 37° C., resulting in formation of an RCA product. A negative control was prepared by mixing and incubating the same reagents without the DNA polymerase.

SERS Detection of RCA Product

One μL of the RCA product and 1 μL of the negative control sample were separately spotted on an aluminum tray and air-dried. Each spot was rinsed with 5 μL of 1×PBS (phosphate buffered saline). The rinse was repeated three times and the aluminum tray was air-dried after the final rinse.

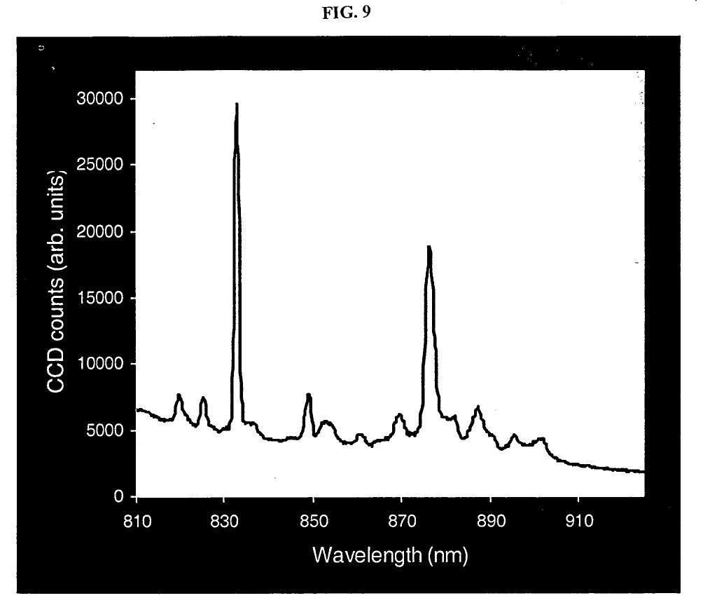

One mL of silver colloid solution prepared as above was diluted with 2 mL of distilled water. Eight microliters of the diluted silver colloid solution was mixed with 2 μL of 0.5 M LiCl and added to the RCA product spot on the aluminum tray. The same solution was added to the negative control spot. The Raman signals were collected as disclosed above. As demonstrated in FIG. 9, an RCA product was detectable by SERS, with emission peaks at about 833 and 877 nm. Under the conditions of this protocol, with an LiCl enhancer, the signal strength from the adenine moieties is stronger than those for guanine, cytosine and thymine. The negative control (not shown) showed that the Raman signal was specific for the RCA product, as no signal was observed in the absence of amplification.

### Example 5

### Exonuclease Digestion of Nucleic Acids

Exonuclease treatment is performed according to Sauer et al. (J. Biotech. 86:181-201, 2001). Single nucleic acid molecules labeled on the 5′ end with biotin are prepared by PCR amplification of a nucleic acid template, using a 5′-biotinylated oligonucleotide primer. A cone-shaped 3 μm single-mode optical fiber (SMC-A0630B, Laser Components GmbH, Olching, Germany) is prepared. The glass fiber is chemically etched with HF to form a sharp tip. After coating with 3-mercaptopropyltrimethoxysilane, the tip is treated with γ-maleinimidobutyric acid N-hydroxysuccinamide (GMBS). The tip of the fiber is activated with streptavidin and allowed to bind to the biotinylated DNA. Unbound DNA is removed by washing.

A fiber containing a single molecule of bound DNA is inserted into a PDMS reaction chamber attached to a 5 μm microchannel. Exonuclease I is added to the reaction chamber to initiate cleavage of the ssDNA. The exonuclease is confined to the reaction chamber by use of an optical trap (e.g. Walker et al., FEBS Lett. 459:39-42, 1999; Bennink et al., Cytometry 36:200-208, 1999; Mehta et al., Science 283:1689-95, 1999; Smith et al., Am. J. Phys. 67:26-35, 1999). Optical trapping devices are available from Cell Robotics, Inc. (Albuquerque, N. Mex.), S+L GmbH (Heidelberg, Germany) and P.A.L.M. Gmbh (Wolfratshausen, Germany). Nucleoside monophosphates are released by exonuclease digestion and transported past a Raman detector, as disclosed in Example 2, by microfluidic flow. The nucleotides in solution are focused within the laser excitation and detection volume through the use of hydrodynamic focusing. A 90 μM concentration of LiCl is added to the detection mixture, and the microfluidic channel in the vicinity of the detector is packed with silver nanoparticles prepared according to Lee and Meisel (1982). Single nucleotides are detected as they flow past the Raman detector, allowing determination of the nucleic acid sequence.

All of the METHODS and APPARATUS disclosed and claimed herein can be made and used without undue experimentation in light of the present disclosure. It will be apparent to those of skill in the art that variations may be applied to the METHODS and APPARATUS described herein without departing from the concept, spirit and scope of the claimed subject matter. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the claimed subject matter.

What is claimed is:

1. A method comprising:
a) sequentially removing nucleotides from one end of at least one nucleic acid molecule;
b) moving the nucleotides through a channel packed with nanoparticles;
c) identifying one or more nucleotides by Raman spectroscopy; and
d) characterizing the nucleic acid.

2. The method of claim 1, wherein the nucleotides are removed from the nucleic acid by exonuclease activity.

3. The method of claim 1, further comprising identifying single nucleotide molecules.

4. The method of claim 3, wherein the nucleotides are unlabeled.

5. The method of claim 3, wherein the nucleotides are labeled.

6. The method of claim 3, further comprising identifying single adenosine nucleotide molecules

7. The method of claim 1, wherein only adenosine and guanosine nucleotides are identified.

8. The method of claim 1, wherein only cytidine and thymidine nucleotides are identified.

9. The method of claim 1, further comprising separating the purine or pyrimidine bases from the nucleotides.

10. The method of claim 9, wherein the separated purine or pyrimidine bases are identified by Raman spectroscopy.

11. The method of claim 1, wherein a single nucleic acid molecule is sequenced.

12. The method of claim 1, wherein the nucleotides are identified by surface enhanced Raman spectroscopy (SERS), surface enhanced resonance Raman spectroscopy (SERRS) and/or coherent anti-Stokes Raman spectroscopy (CARS).

13. The method of claim 1, wherein the channel is a nanochannel or microchannel.

14. The method of claim 1, further comprising identifying the nucleic acid.

15. The method of claim 1, further comprising sequencing the nucleic acid.

16. The method of claim 1, further comprising identifying a single nucleotide polymorphism in the nucleic acid.

17. A method comprising:
a) preparing a nucleic acid comprising labeled nucleotides;
b) sequentially removing nucleotides from one end of the nucleic acid;
c) moving the nucleotides through a channel packed with nanoparticles;
d) identifying one or more nucleotides by Raman spectroscopy; and
e) characterizing the nucleic acid.

18. The method of claim 17, wherein each type of nucleotide is labeled with a distinguishable Raman label.

19. The method of claim 18, wherein only pyrimidine nucleotides are labeled.

20. The method of claim 18, wherein only purine nucleotides are labeled.

21. The method of claim 17, wherein single nucleotide molecules are identified.

22. The method of claim 17, further comprising identifying single adenosine nucleotide molecules.

23. The method of claim 17, further comprising separating the nucleotides from the nucleic acid.

24. The method of claim 23, further comprising imposing an electric field to move the nucleotides through the channel.

25. The method of claim 12, further comprising recording the time at which each nucleotide passes through said channel.

26. An apparatus comprising:
a) a reaction chamber;
b) a first channel in fluid communication with the reaction chamber;
c) a second channel in fluid communication with the first channel;
d) a multiplicity of nanoparticles packed in the second channel; and
e) a Raman detector operably coupled to the nanoparticle packed channel.

27. The apparatus of claim 26, wherein the apparatus is capable of detecting single nucleotide molecules.

28. The apparatus of claim 26, further comprising a first electrode and a second electrode to move nucleotides from the first channel into the second channel.

29. The apparatus of claim 20, wherein the first channel is a microfluidic channel.

30. The apparatus of claim 20, wherein the second channel is a nanochannel or a microchannel.
